(19) United States
(12) Patent Application Publication
(10) Pub. No.: US 2025/0345204 A1
(43) Pub. Date: Nov. 13, 2025

(54) EYE COMPRESSES ENABLING PROLONGED THERMAL TREATMENT

(71) Applicant: Vivian Surya Buehler, Gales Creek, OR (US)

(72) Inventor: Vivian Surya Buehler, Gales Creek, OR (US)

(21) Appl. No.: 19/202,738

(22) Filed: May 8, 2025

Related U.S. Application Data
(60) Provisional application No. 63/644,388, filed on May 8, 2024.

(51) Int. Cl. A61F 7/02; A61F 7/00

(52) U.S. Cl. CPC A61F 7/02; A61F 7/007; A61F 2007/0004; A61F 2007/008; A61F 2007/0219; A61F 2007/0231

(57) ABSTRACT

Eye compresses including a strap, a patch, and an electric heating element. The strap is configured to selectively couple to a head of a user. The patch is coupled to the strap and configured to cover an eye of the user when the strap is selectively coupled to the head of the user. The patch defines a pocket with an interior and a pocket opening. The electric heating element is disposed in the interior of the pocket. The electric heating element is configured to selectively generate heat when electrically coupled to a power source.

### CROSS REFERENCE TO RELATED APPLICATIONS

This application claims priority to copending U.S. Application, Ser. No. 63/644,388 filed on May 8, 2024 which is hereby incorporated by reference for all purposes.

### BACKGROUND

The present disclosure relates generally to compresses. In particular, eye compresses enabling prolonged thermal treatment are described.

Eye strain, fatigue, and dryness (collectively “ocular discomfort”) are conditions endured by many people. People who spend extended time in front of computer monitors, television screens, and handheld device screens have an increased risk of ocular discomfort. Thus, people who utilize computers, handheld devices, and televisions while working, playing computer games, browsing the internet, or engaging in social media tend to have ocular discomfort with increased prevalence.

Amblyopia treatments and other ocular treatment methodologies are based on the understanding that covering one eye while the other eye is uncovered provides various vision and ocular health benefits. For example, selectively utilizing one eye while allowing the other eye to rest is known to enhance ocular motor and vision alignment between the eyes. Selectively using one eye while covering the other eye is also believed to sharpen vision. Patching a normally functioning eye is a recognized treatment to address “lazy eye” conditions in the other eye.

Compresses are known to treat wounds and ocular issues by applying a combination of pressure and a temperature gradient to a localized area on the body. Eye compresses work by heating or cooling the eye and the tissue surrounding the eye concurrently with slight pressure applied to the eye region. Eye compresses can take the form of an eye patch, a wrap, or a wad of material.

Conventional eye compresses are not fully satisfactory. For example, conventional eye compresses often require soaking the compress in hot or cold water, which is inconvenient and leads to water undesirably dripping from the compress. The wet compress can foster bacteria and cause ocular complications, such as eye irritation and infections.

A key limitation of conventional compresses is that the hot or cold treatment supplied is temporary-often lasting only a relatively short duration. The short duration of hot or cold treatment necessitates stopping treatment and heating or cooling the compress again to enable further treatment. The disjointed treatment can reduce the effectiveness of the treatment and limit one's ability to engage in other activities.

It would be desirable to have an eye compress that enabled prolonged hot and cold treatment. It would be beneficial to have an eye compress that enabled easily switching between hot and cold treatments. It would be advantageous to have an eye compress that did not require soaking the compress in hot or cold water given the unsanitary conditions and eye complications that can result from wet eye compresses.

It would be additionally beneficial if the eye compress enabled one to engage in other activities while utilizing the compress. If an eye compress existed that allowed for prolonged heat treatment without requiring the user to hold the compress in place, one could use the compress to improve vision acuity, ocular motor alignment, and vision alignment concurrently with treating an eye. For example, one could proactively use such a novel eye compress while engaged in an activity, such as computer gaming, to rest and treat ocular discomfort in one eye while improving his or her performance in the activity through vision acuity and alignment improvements.

Thus, there exists a need for eye compresses that improve upon and advance the design of known eye compresses and eye patches. Examples of new and useful eye compresses relevant to the needs existing in the field are discussed below.

Disclosure relevant to eye compresses is included in the following U.S. Patent References: U.S. Pat. Nos. 11,389,331 and 8,636,786. The complete disclosures of the listed patents are herein incorporated by reference for all purposes.

### SUMMARY

The present disclosure is directed to eye compresses including a strap, a patch, and an electric heating element. The strap is configured to selectively couple to a head of a user.

The patch is coupled to the strap and configured to cover an eye of the user when the strap is selectively coupled to the head of the user. The patch defines a pocket with an interior and a pocket opening.

The electric heating element is disposed in the interior of the pocket. The electric heating element is configured to selectively generate heat when electrically coupled to a power source.

### BRIEF DESCRIPTION OF THE DRAWINGS

FIG. 1 is a perspective view of a first example of an eye compress worn by a user over an eye.

FIG. 2 is a top perspective view of the eye compress shown in FIG. 1 depicting a pocket open to receive an electric heating element, a thermal insert, or a weight insert.

FIG. 3 is a front view of an electric heating element configured to insert in the pocket of the eye compress shown in FIG. 1.

FIG. 4 is a front view of a thermal insert configured to insert in the pocket of the eye compress shown in FIG. 1.

FIG. 5 is a front view of a weight insert configured to insert in the pocket of the eye compress shown in FIG. 1.

### DETAILED DESCRIPTION

The disclosed eye compresses will become better understood through review of the following detailed description in conjunction with the figures. The detailed description and figures provide merely examples of the various inventions described herein. Those skilled in the art will understand that the disclosed examples may be varied, modified, and altered without departing from the scope of the inventions described herein. Many variations are contemplated for different applications and design considerations; however, for the sake of brevity, each and every contemplated variation is not individually described in the following detailed description.

Throughout the following detailed description, examples of various eye compresses are provided. Related features in the examples may be identical, similar, or dissimilar in different examples. For the sake of brevity, related features will not be redundantly explained in each example. Instead, the use of related feature names will cue the reader that the feature with a related feature name may be similar to the related feature in an example explained previously. Features specific to a given example will be described in that particular example. The reader should understand that a given feature need not be the same or similar to the specific portrayal of a related feature in any given figure or example.

### Definitions

The following definitions apply herein, unless otherwise indicated.

“Substantially” means to be more-or-less conforming to the particular dimension, range, shape, concept, or other aspect modified by the term, such that a feature or component need not conform exactly. For example, a “substantially cylindrical” object means that the object resembles a cylinder, but may have one or more deviations from a true cylinder.

“Comprising,” “including,” and “having” (and conjugations thereof) are used interchangeably to mean including but not necessarily limited to, and are open-ended terms not intended to exclude additional elements or method steps not expressly recited.

Terms such as “first”, “second”, and “third” are used to distinguish or identify various members of a group, or the like, and are not intended to denote a serial, chronological, or numerical limitation.

“Coupled” means connected, either permanently or releasably, whether directly or indirectly through intervening components.

“Communicatively coupled” means that an electronic device exchanges information with another electronic device, either wirelessly or with a wire-based connector, whether directly or indirectly through a communication network.

“Controllably coupled” means that an electronic device controls operation of another electronic device.

### Eye Compresses Enabling Prolonged Thermal Treatment

With reference to the figures, eye compresses enabling prolonged thermal treatment will now be described. The novel eye compresses discussed herein function to selectively heat or cool an eye and surrounding tissue for a prolonged period of time. Further, the novel compresses described below function to cover one eye to enable enhancing vision acuity, motor alignment, and vision alignment while resting the other eye.

The reader will appreciate from the figures and description below that the presently disclosed novel eye compresses address many of the shortcomings of conventional eye compresses. For example, the novel eye compresses enable prolonged hot and cold treatment rather than the short duration thermal treatments possible with conventional eye compresses. Beneficially, the novel eye compresses enable easily and quickly switching between hot and cold treatments. Advantageously, the novel eye compresses do not require soaking them in hot or cold water, which avoids the unsanitary conditions and eye complications that can result from wet eye compresses.

An additional benefit of the novel eye compresses is that they enable one to engage in other activities while utilizing them. The novel eye compresses allow for prolonged thermal treatment without requiring the user to hold the compress in place on the eye, which allows for improving vision acuity, ocular motor alignment, and vision alignment concurrently with treating an eye. For example, one can proactively use the novel eye compresses while engaged in an activity, such as computer gaming, to rest and treat ocular discomfort in one eye while improving his or her performance in the activity though vision acuity and alignment improvements.

### Eye Compress Embodiment One

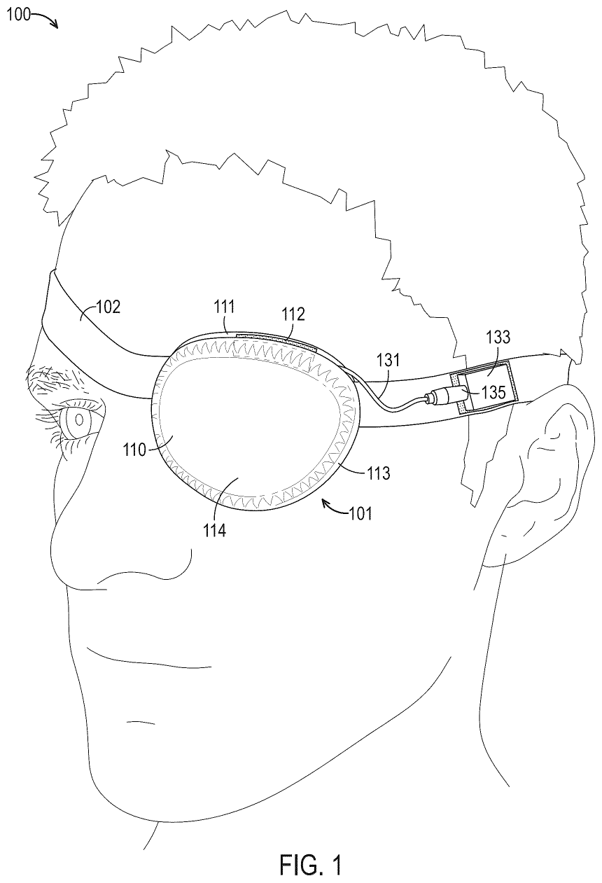

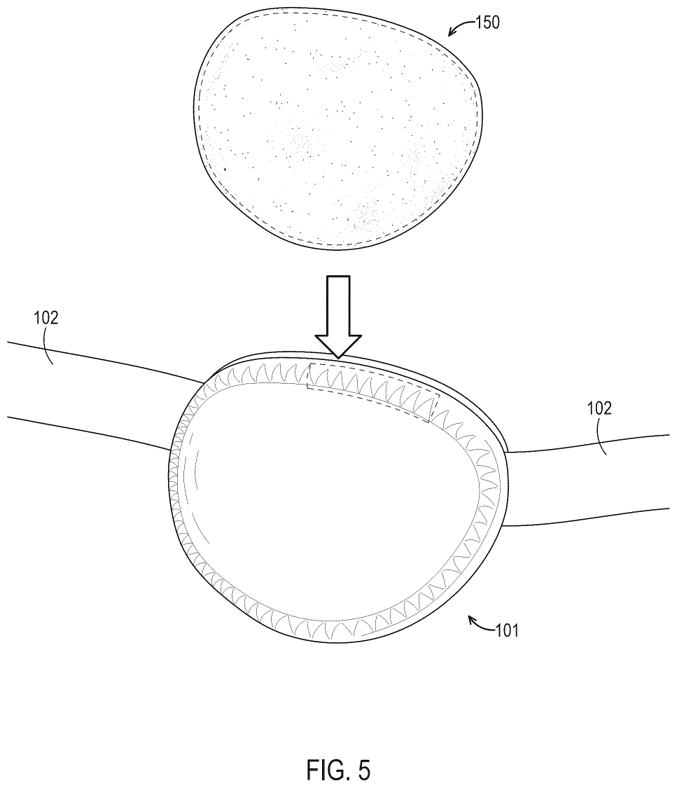

With reference to FIGS. 1-5, a first example of an eye compress, eye compress 100, will now be described. Eye compress 100 is configured to be worn over a selected eye and to remain in place indefinitely until selectively removed. A user does not have hold eye compress 100 in place and can instead use both hands for other activities, such as controlling a game controller or mouse and keyboard.

Eye compress 100 is configured to apply heat to a selected eye and surrounding tissue to provide hot therapy. The selected eye and surrounding tissue is referred to herein as a target eye region. Eye compress 100 can supply heat to a target eye region indefinitely via an electric heating element.

Eye compress 100 is also configured to extract heat from a target eye region to provide cold therapy. A user can easily opt for hot therapy or cold therapy when using eye compress 100 by selectively activing an electric heating element, utilizing a heated thermal insert, or utilizing a cooled thermal insert.

In the example shown in FIGS. 1-5, eye compress 100 includes a patch 101, a strap 102, an electric heating element 103, a thermal insert 140, and a weight insert 150. The components of eye compress 100 are discussed in the sections below.

In some examples, the eye compress does not include one or more features included in eye compress 100. For example, some eye compress examples do not include thermal inserts or weight inserts. In other examples, the eye compress includes additional or alternative features.

### Patch

As shown in FIG. 1, patch 101 functions to cover an eye and surrounding tissue of a user. Patch 101 also serves to receive and support one or more of electric heating element 103, thermal insert 140, and weight insert 150.

As shown in FIGS. 1, 2, 4, and 5, patch 101 is mounted to strap 102. Patch 101 and strap 102 cooperate to selectively position patch 101 over a selected eye. As shown in FIG. 1, strap 102 supports patch 101 in an eye covering position when strap 102 extends over and couples to a user's head.

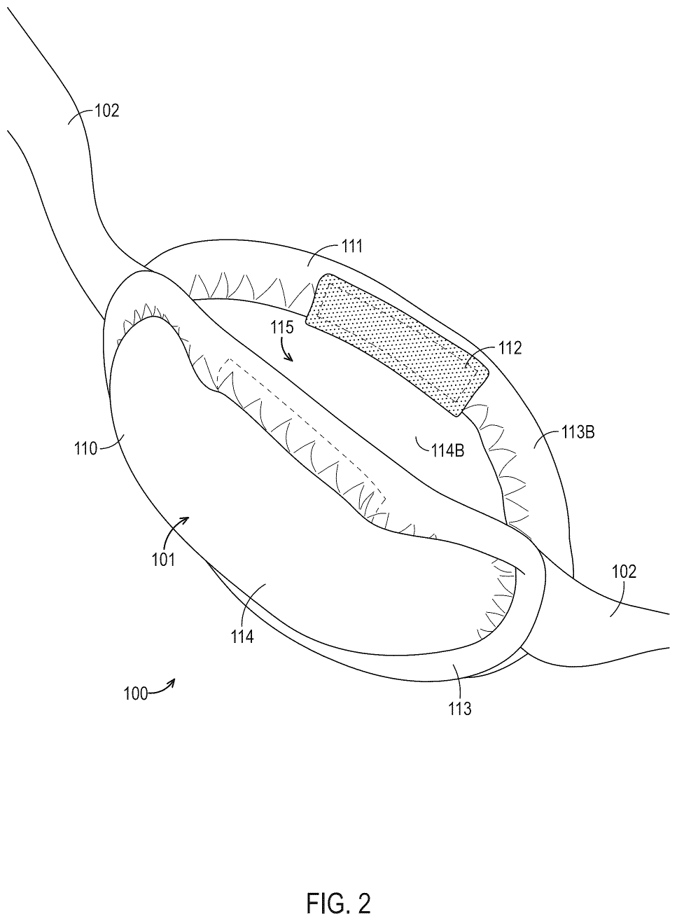

FIG. 2 demonstrates that patch 101 defines a pocket 115 with pocket walls 110, an opening 111, and a closure member 112. Electric heating element 103, thermal insert 140, and/or weight insert 150 may be inserted into pocket 115 through opening 111 and supported therein by pocket walls 110. When desired, electric heating element 103, thermal insert 140, and/or weight insert 150 may be easily removed from pocket 115 through opening 111.

Closure member 112 functions to selectively close opening 111 to retain electric heating element 103, thermal insert 140, and/or weight insert 150 in pocket 115 between pocket walls 110. In the present example, closure member 112 is a hook-and-loop fastener. In other examples, the closure member is a mechanical fastener, such as a button, clip, or clasp. In some examples, the closure member is a magnetic coupler.

In the present example, patch 101 is shaped and sized to cover an eye and surrounding tissue to a selected extent. However, the size and shape of the patch may be varied to cover more or less area of a target eye region.

As shown in FIG. 1, patch 101 is formed from fabric with defined border portions 113 and central expanse cushion portions 114. An exterior surface of central expanse cushion portion 114 facing the user's eye is a soft fabric material to provide a comfortable interface between patch 101 and the eye of the user. The patch may be formed from any suitable material or combination of materials, including polymers and composite materials.

With reference to FIG. 2, the reader can see that patch 101 includes two border portions 113A and 113B and two central expanse cushion portions 114A and 114B. Border portion 113A extends around a periphery of patch 101 on a first side of patch 101, and border portion 113B extends around the periphery of patch 101 on a second side of patch 101 opposite the first side. Expanse cushion portion 114A is bounded by border portion 113A, and expanse cushion portion 114B is bounded by border portion 113B.

FIG. 2 demonstrates that border portion 113A and border portion 113B are partially sewn together. As shown in FIG. 2, partially sewing border portions 113A and 113B together defines pocket 115. As further shown in FIG. 2, pocket opening 111 is defined where border portions 113A and 113B are not sewn together; that is, pocket opening 111 is defined where there is a gap in the stitching between border portions 113A and 113B.

### Strap

As shown in FIGS. 1, 2, 3, and 4, strap 102 functions to support patch 101 and other components of eye compress 100 contained within patch 101. The reader can see in FIG. 1 that strap 102 is configured to wrap around and comfortably couple to a person's head. Patch 101 is coupled to strap 102 and hangs from strap 102 when strap 102 is secured to a person's head.

In the example shown in FIGS. 1, 2, 3, and 4, strap 102 is formed from an elastic material. However, the strap need not be elastic to function. For example, the strap may be inelastic and secure to the user's head when adjusted to maintain a selected amount of tension. The strap may be formed from any currently known or later developed material suitable for securing to a person's body and supporting the patch.

The size and shape of the strap may vary in different examples. In some examples, the strap is a universal size appropriate for men, women, and children with various head sizes. In certain examples, the strap will be selected to correspond to a selected head size or range of head sizes, such as small, medium, and large size ranges. The strap may additionally or alternatively be selected to correspond to head sizes typical for different genders or age ranges. The size of the strap may be fixed or adjustable.

### Electric Heating Element

Electric heating element 103 serves to deliver heat through patch 101 to a selected eye region to provide hot therapy to the selected eye region. Notably, electric heating element 103 is configured to deliver heat indefinitely as long as electric heating element 103 is supplied with electricity.

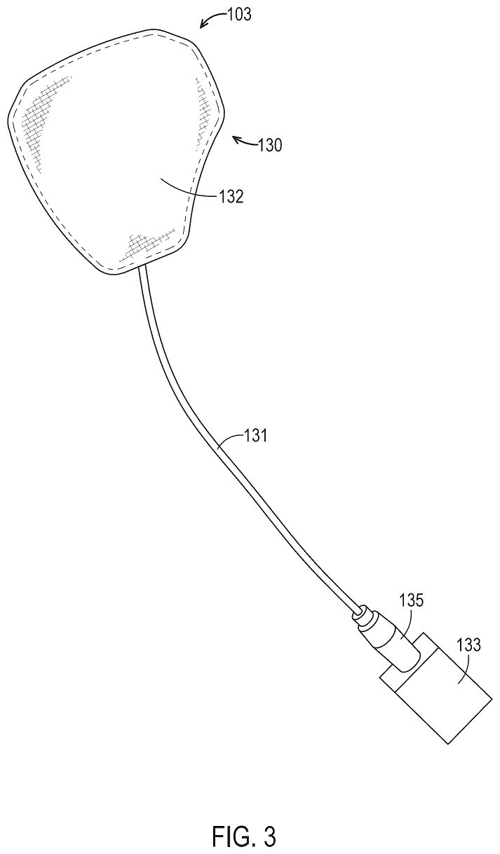

As shown in FIG. 3, electric heating element 103 includes a heating pad 130, a power cord 131, and a battery 133. In the present example, power cord 131 delivers stored electricity from battery 133 to heating pad 130. In other examples, the power cord couples to a power source in the form of a wall outlet, an electronic device, or a vehicle power connector.

Power cord 131 includes a power connector 135 configured to electrically couple to a power source, such as a wall outlet, battery 133, or an electronic device. In the example shown in FIGS. 1 and 3, power connector 135 is a barrel plug. However, the power connector may be any currently known or later developed type of power connector, including HDMI™ types A, B, and C in mini, micro, and standard variants, pin connectors, plug connectors, and wireless power arrangements.

Heating pad 130 is configured to convert electricity supplied by battery 133 or other power source via power cord 131 into heat via an internal heating element (not pictured). The heating pad may utilize any currently known or later developed technology or technique for generating heat from electricity, including electric resistance heaters, heat pumps, and radiative heating elements.

Battery 133 is a rechargeable, lithium-ion battery. However, the battery need not be rechargeable and may have different chemical formulations. The battery may be any currently known or later developed type of battery suitable for powering electric heating elements.

As demonstrated in FIG. 1, battery 133 is supported on strap 102. Battery 133 mounts to strap 102 via a hook-and-loop fastener. In other examples, the battery mounts to the strap with mechanical fasteners, such as a clip or a snap; adhesives; magnetic couplers; or stitching. In some examples, the battery is disposed in the pocket of the patch.

FIG. 3 makes apparent that heating pad 130 includes a thermal buffer member in the form of a protective cover 132 to isolate the internal heating element. Protective cover 132 is disposed between the internal heating element and the eye of the user when strap 102 is selectively coupled to the head of the user. Protective cover 132 avoids exposing patch 101, a user, or nearby objects to elevated temperatures that may be present at the internal heating element. Further, protective cover 132 avoids external contaminants interfering with the internal heating element as it operates.

### Thermal Insert

Thermal insert 140 functions to provide hot or cold therapy through patch 101 to a selected eye region. Cold therapy is administered by cooling thermal insert 140 before inserting it into pocket 115 of patch 101 and bringing patch 101 into contact with the target eye region. When administering cold therapy, thermal insert 140 is effective to reduce the temperature of the target eye region.

Hot therapy with thermal insert 140 is administered by heating thermal insert 140 before inserting it into pocket 115 of patch 101 and bringing patch 101 into contact with the target eye region. When administering hot therapy, thermal insert 140 is effective to increase the temperature of the target eye region.

Thermal insert 140 may be primarily utilized to extract heat from the target eye region because electric heating element 103 provides a means to supply heat indefinitely in contrast to thermal insert 140 that delivers heat for a finite period of time. However, thermal insert 140 could be heated to deliver heat to the target eye region instead. It is preferred to utilize electric heating element 103 for delivering heat to the target eye region because the heat therapy can last indefinitely, but using thermal insert 140 instead for hot therapy may be necessary when a power source is not available.

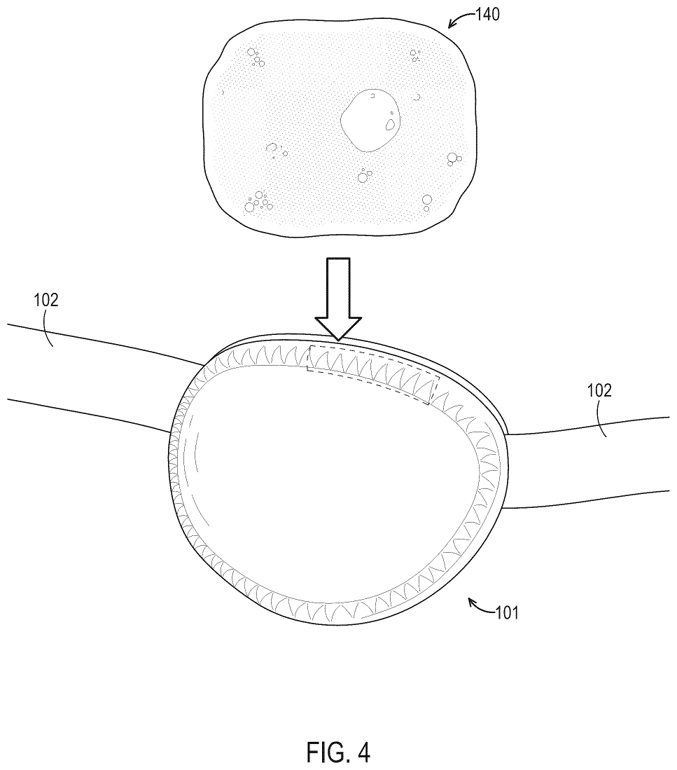

As apparent from FIG. 4, thermal insert 140 is a gel pack sized to fit inside pocket 115 of patch 101. However, the thermal insert may be any currently known or later developed fluid packet or other item suitable for storing and releasing thermal energy. In some examples, the fluid packet of thermal insert is filled with water or other heat-transfer fluid beyond gel materials.

### Weight Insert

Weight insert 150 serves to selectively add weight to patch 101 to increase the pressure patch 101 applies to a target eye region when worn. Increased pressure may intensify the hot or cold therapy delivered by eye compress 100. Increased pressure may also beneficially modify the blood flow to the target eye region to promote healing, recuperation, or alleviation of eye discomfort symptoms.

Weight insert 150 is a packet sized to fit inside pocket 115 of patch 101 that contains a fill material. The amount of fill material contained in weight insert 150 has a selected aggregate weight effective to modify blood flow to the target eye region to a selected extent. In some examples, the weight insert is configured for the user to add a desired amount of fill material to achieve a desired aggregate weight for the weight insert. In some examples, multiple weight inserts are included in the eye compress.

A variety of fill materials may be contained in the weight insert. Weight insert 150 contains sand as a fill material. The fill material may be crystals of various kinds. Sand, polymers, liquids, composite materials, and mixtures of materials may all be used as fill material for the weight insert.

Eye compress 100 includes a single weight insert 150, but the number of weight inserts included in the eye compress will vary in different examples. Some examples include multiple weight inserts to allow a range of weights to be inserted into the patch. In select examples, the eye compress does not include any weight inserts.

The disclosure above encompasses multiple distinct inventions with independent utility. While each of these inventions has been disclosed in a particular form, the specific embodiments disclosed and illustrated above are not to be considered in a limiting sense as numerous variations are possible. The subject matter of the inventions includes all novel and non-obvious combinations and subcombinations of the various elements, features, functions and/or properties disclosed above and inherent to those skilled in the art pertaining to such inventions. Where the disclosure or subsequently filed claims recite “a” element, “a first” element, or any such equivalent term, the disclosure or claims should be understood to incorporate one or more such elements, neither requiring nor excluding two or more such elements.

Applicant(s) reserves the right to submit claims directed to combinations and subcombinations of the disclosed inventions that are believed to be novel and non-obvious. Inventions embodied in other combinations and subcombinations of features, functions, elements and/or properties may be claimed through amendment of those claims or presentation of new claims in the present application or in a related application. Such amended or new claims, whether they are directed to the same invention or a different invention and whether they are different, broader, narrower or equal in scope to the original claims, are to be considered within the subject matter of the inventions described herein.

1. An eye compress, comprising:
a strap configured to selectively couple to a head of a user;
a patch coupled to the strap and configured to cover an eye of the user when the strap is selectively coupled to the head of the user, the patch defining a pocket with an interior and a pocket opening; and
an electric heating element disposed in the interior of the pocket, the electric heating element configured to selectively generate heat when electrically coupled to a power source.

2. The eye compress of claim 1, further comprising a thermal insert selectively disposed in the interior of the pocket.

3. The eye compress of claim 2, wherein the thermal insert includes:
a fluid packet; and
a heat transfer fluid contained within the fluid packet.

4. The eye compress of claim 3, wherein the heat transfer fluid is a gel.

5. The eye compress of claim 2, wherein the thermal insert is cooled prior to being selectively disposed in the interior of the pocket.

6. The eye compress of claim 5, wherein the thermal insert is effective to reduce the temperature of one or more of the eye of the user and tissue surrounding the eye of the user when the patch covers the eye of the user and the thermal insert is disposed in the interior of the pocket after being cooled.

7. The eye compress of claim 1, further comprising a weight insert.

8. The eye compress of claim 7, wherein the weight insert includes:
a weight packet; and
fill material contained within the weight packet.

9. The eye compress of claim 7, wherein the weight insert is effective to reduce blood flow to one or more of the eye of the user and tissue surrounding the eye of the user when the patch covers the eye of the user and the weight insert is disposed in the interior of the pocket.

10. The eye compress of claim 1, wherein the electric heating element is configured to selectively generate heat indefinitely when electrically coupled to a power source.

11. The eye compress of claim 1, further comprising a battery electrically coupled to the electric heating element, the battery defining a power source.

12. The eye compress of claim 11, wherein the battery is supported on the strap outside the pocket.

13. The eye compress of claim 1, wherein the patch includes a closure member configured to selectively close the pocket opening.

14. The eye compress of claim 13, wherein the closure member is a hook-and-loop fastener.

15. The eye compress of claim 1, wherein the electric heating element includes:
a heating device configured to selectively generate heat when powered with electricity; and
a buffer member configured to thermally buffer the heating device.

16. The eye compress of claim 15, wherein the buffer member is disposed between the heating device and the eye of the user when the strap is selectively coupled to the head of the user.

17. The eye compress of claim 1, wherein:
the patch includes an exterior surface proximate the eye of the user when the strap is selectively coupled to the head of the user; and
the exterior surface is soft to provide a comfortable interface between the patch and the eye of the user.

18. The eye compress of claim 1, wherein the patch includes:
a first border extending around a periphery of the patch on a first side of the patch;
a second border extending around the periphery of the patch on a second side of the patch opposite the first side;
a first expanse bounded by the first border; and
a second expanse bounded by the second border.

19. The eye compress of claim 18, wherein:
the first border and the second border are partially sewn together to define the pocket; and
the pocket opening is defined where the first border is not sewn to the second border.

20. The eye compress of claim 1, wherein the strap is formed from an elastic material.
